(19) United States
(12) Patent Application Publication
(10) Pub. No.: US 2020/0107991 A1
(43) Pub. Date: Apr. 9, 2020

(54) AUXILIARY ACUPUNCTURE SYSTEM

(71) Applicant: I-SHOU UNIVERSITY, Kaohsiung (TW)

(72) Inventors: Chih-Yu Wang, Kaohsiung (TW); Shu-Chen Chang, Kaohsiung (TW); Che-Chang Kuo, Kaohsiung (TW); Chih-Er Wu, Kaohsiung (TW)

(21) Appl. No.: 16/225,037

(22) Filed: Dec. 19, 2018

(30) Foreign Application Priority Data
Oct. 3, 2018 (TW) 107134958

(51) Int. Cl. A61H 39/08; G16H 20/30; A61H 39/04; A61H 39/02

(52) U.S. Cl. CPC A61H 39/08; A61H 39/02; A61H 39/04; G16H 20/30

(57) ABSTRACT

An auxiliary acupuncture system includes an auxiliary acupuncture system includes a human machine interface, a storage unit and a processing unit. The human machine interface is configured to receive at least one symptom of an illness. The storage unit is configured to store acupoint information. The acupoint information contains a name and a therapeutic function of each of a plurality of acupoints. The plurality of acupoints is divided into a plurality of sets of acupoints. The processing unit is coupled with the human machine interface and the storage unit, retrieves at least one of the plurality of sets of acupoints from the acupoint information which has a therapeutic effect for the at least one symptom of the illness, and displays the at least one of the plurality of sets of acupoints on the human machine interface.

### CROSS REFERENCE TO RELATED APPLICATION

The application claims the benefit of Taiwan application serial No. 107134958, filed on Oct. 3, 2018, and the entire contents of which are incorporated herein by reference.

### BACKGROUND OF THE INVENTION

### 1. Field of the Invention

The present invention generally relates to an auxiliary medical system and, more particularly, to an auxiliary acupuncture system that can analyze which acupoints should be used for treatment for the illness of the patient according to at least one symptom of the illness.

### 2. Description of the Related Art

There are a number of acupoints in the human body. Among all of the recognized acupoints, there are 361 acupoints for twelve main meridians and there are 152 extra ordinary points not belonging to the twelve main meridians. In addition, there are also 1500 new acupoints recognized and the number of the recognized acupoints is still increasing.

Due to the increasing number of the acupoints where different acupoints have different therapeutic functions but a single acupoint can have more than one therapeutic function, the inexperienced acupuncturist possibly does not know all of the combinations of the acupoints that have a therapeutic effect for the same symptom. As a result, the treatment of the acupuncturist lacks variation and is insufficient.

In light of the problem, it is necessary to provide an auxiliary acupuncture system which allows the acupuncturist to deal with the same symptom of the illness of the patient through various combinations of the acupoints, thus providing different approaches of treatment.

### SUMMARY OF THE INVENTION

It is therefore the objective of this invention to provide an auxiliary acupuncture system which can analyze the symptom(s) of the illness of the patient and accordingly determine various combinations of the acupoints which have a therapeutic effect for the illness of the patient. This permits the acupuncturist to treat the patient with different approaches.

In an aspect, an auxiliary acupuncture system includes an auxiliary acupuncture system includes a human machine interface, a storage unit and a processing unit. The human machine interface is configured to receive at least one symptom of an illness. The storage unit is configured to store acupoint information. The acupoint information contains a name and a therapeutic function of each of a plurality of acupoints. The plurality of acupoints is divided into a plurality of sets of acupoints. The processing unit is coupled with the human machine interface and the storage unit, retrieves at least one of the plurality of sets of acupoints from the acupoint information which has a therapeutic effect for the at least one symptom of the illness, and displays the at least one of the plurality of sets of acupoints on the human machine interface.

Based on the above, the auxiliary medical system according to the invention can obtain at least one set of the acupoints, which has the therapeutic effect for at least one symptom of the illness, from the previously-established acupoint information. Each of the at least one set of the acupoints has a combination of the acupoints different from those of the other sets. Thus, the acupuncturist can select one of the at least one set of the acupoints to perform treatment for at least one symptom of the illness. Furthermore, the auxiliary medical system according to the invention permits the acupuncturist to perform different approaches of treatment on the patient through different combinations of the acupoints. As a result, low therapeutic effect resulting from the lack of the experience of the acupuncturist is avoided, thereby improving the medical quality.

In an example, the human machine interface further receives a medical requirement, and the processing unit retrieves the at least one of the plurality of sets of acupoints which has the therapeutic effect for the at least one symptom of the illness and meets the medical requirement. Thus, the auxiliary medical system according to the invention can select the suitable acupoints according to the medical requirement.

In the example, the at least one symptom of the illness includes a plurality of symptoms of the illness. The medical requirement indicates a minimum number of needle insertions. The processing unit selects and displays one of the at least one of the plurality of sets of acupoints which has a minimum number of acupoints on the human machine interface. Thus, if the patient has a fear of acupuncture, the auxiliary medical system according to the invention can reduce the number of the needle insertions under a certain therapeutic effectiveness, thus reducing the pressure of the patient.

In the example, the acupoint information contains an effectiveness index of each of the plurality of acupoints in treatment for each of the at least one symptom of the illness. Thus, the auxiliary medical system according to the invention can select the acupoint according to the effectiveness indices of the plurality of acupoints.

In the example, the medical requirement indicates a highest therapeutic effectiveness for one of the at least one symptom of the illness under a same number of needle insertions. Each of the at least one of the plurality of sets of acupoints includes at least one of the plurality of acupoints which has the therapeutic effect for the symptom of the illness. The effectiveness indices of the at least one of the plurality of acupoints are added to yield a sum for each of the at least one of the plurality of sets of acupoints. The highest therapeutic effectiveness is the sum of one of the at least one of the plurality of sets of acupoints which is the largest. The one of the at least one of the plurality of sets of acupoints is output to the human machine interface. Thus, the auxiliary medical system according to the invention can enhance the therapeutic strength on the acupoints associated with one of the plurality of symptoms of the illness that has a higher degree of severity.

In the example, the at least one symptom of the illness includes a plurality of symptoms of the illness. The medical requirement indicates a highest weight in therapeutic effectiveness for the plurality of symptoms of the illness under a same number of needle insertions. Each of the at least one of the plurality of sets of acupoints includes at least one of the plurality of acupoints. The effectiveness indices of the at least one of the plurality of acupoints are added to yield a weight in therapeutic effectiveness for the plurality of symptoms of the illness for each of the at least one of the plurality of sets of acupoints. The highest weight in therapeutic effectiveness is the weight in therapeutic effectiveness of one of the at least one of the plurality of sets of acupoints which is the highest. The one of the at least one of the plurality of sets of acupoints is output to the human machine interface. Thus, the auxiliary medical system according to the invention can improve the overall therapeutic effect of the plurality of symptoms of the illness.

In the example, the storage unit further stores a human body acupoint distribution image. The processing unit marks the plurality of acupoints on the human body acupoint distribution image and displays the marked human body acupoint distribution image on the human machine interface. Thus, it would be more convenient for the acupuncturist to view the locations of the plurality of acupoints through the human body acupoint distribution image.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

The sole FIGURE shows a systematic diagram of an auxiliary acupuncture system according to an embodiment of the invention.

### DETAILED DESCRIPTION OF THE INVENTION

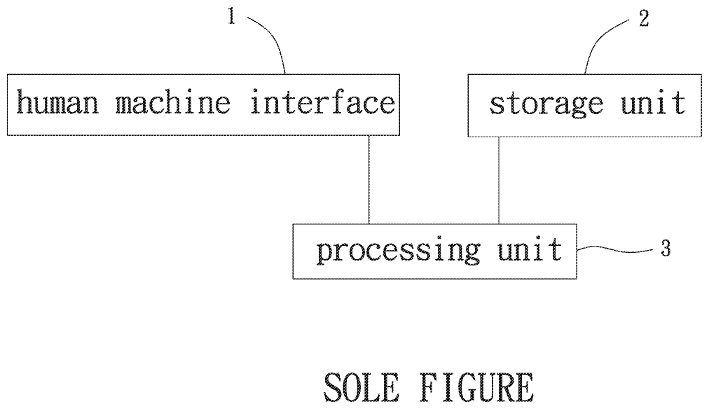

FIG. 1 shows an auxiliary acupuncture system according to an embodiment of the invention. The auxiliary acupuncture system includes a human machine interface 1, a storage unit 2 and a processing unit 3. The storage unit 2 and the processing unit 3 are coupled with the human machine interface 1.

The human machine interface 1 can be any human machine interaction module which provides an input function, an output function and a display function. The human machine interface 1 may be a desk computer or a mobile device with a touch control function. The medical staff can input at least one symptom of the illness of the patient to the human machine interface 1. Preferably, the human machine interface 1 also permits the medical staff to input therapy information regarding the medical requirement. The medical requirement can include, for example, at least one of the options of: (1) the minimum number of the needle insertions, (2) the highest therapeutic effectiveness for one of a plurality of symptoms of the illness under the same number of the needle insertions, and (3) the highest weight in therapeutic effectiveness for a plurality of symptoms of the illness under the same number of the needle insertions.

The storage unit 2 may be any storage medium capable of storing electronic data. In a non-limited example, the storage unit 2 is a hard drive or a memory device. The storage unit 2 is used to store acupoint information associated with the therapeutic function of each of the acupoints. The acupoint information includes the names and therapeutic functions of all of the acupoints of the human body. The acupoint information preferably includes an effectiveness index of each acupoint indicative of how effective the acupoint can be used for treatment for a certain symptom of the illness. The storage unit 2 may further store a human body acupoint distribution image. The human body acupoint distribution image includes at least one of a front head acupoint distribution image, a rear head acupoint distribution image, a chest acupoint distribution image, an abdomen acupoint distribution image, a back acupoint distribution image, an acupoint distribution image of the inner and outer sides of the upper limb, and an acupoint distribution image of the inner and outer sides of the lower limb. However, the above is not used to limit the invention. The human body acupoint distribution image may be generated from a human body image applied with a bone measurement mechanism, as it can be readily appreciated by the person skilled in the art.

The processing unit 3 is coupled with the human machine interface 1 and the storage unit 2 and may be any circuit having a data processing function, a signal generation function and a signal control function. For example, the processing unit 3 may be a processor, a microcontroller, a digital signal processor, a logic circuit or an application-specific integrated circuit (ASIC). However, the processing unit 3 is not limited to the above. In this embodiment, the processing unit 3 is a processor. As the total acupoints of the human body are categorized into a plurality of sets of acupoints, the processing unit 3 can display at least one set of the acupoints on the human machine interface 1 for which a certain symptom or symptoms of the illness can be treated.

Specifically, the processing unit 3 can retrieve at least one set of the acupoints from the therapy information which has a therapeutic effect for the symptom(s) of the illness. The processing unit 3 displays the at least one set of the acupoints on the human machine interface 1. Preferably, when a medical requirement is input to the human machine interface 1, the processing unit 3 can retrieve at least one of the plurality of sets of acupoints from the therapy information which has a therapeutic effect for the symptom(s) of the illness and meets the medical requirement. For example, when the patient has more than one symptom and the medical requirement requires the use of a minimum number of the needle insertions, the processing unit 3 can retrieve one set of the acupoints from the at least one set of the acupoints which has a minimum number of the acupoints. Then, the processing unit 3 displays the set of the acupoints which has the minimum number of the acupoints on the human machine interface 1. As stated above, the acupoint information includes an effectiveness index of each acupoint indicative of how effective the acupoint can be used for treatment for a symptom of the illness. In this regard, when the medical requirement requires the highest therapeutic effectiveness for one of a plurality of symptoms of the illness under the same number of the needle insertions, the processing unit 3 can add the effectiveness indices of those acupoints of each of the at least one set which have a therapeutic effect for the symptom of the illness, thereby obtaining a sum of the effectiveness indices for each of the at least one set of the acupoints. Finally, one of the at least one set of the acupoints which has a largest sum of the effectiveness indices is output to the human machine interface 1 by the processing unit 3. In another case where the medical requirement requires the highest weight in therapeutic effectiveness for the symptoms of the illness under the same number of the needle insertions, the processing unit 3 adds the effectiveness indices of those acupoints of each of the at least one set which have a therapeutic effect for the symptoms of the illness, thereby obtaining a weight in therapeutic effectiveness for the symptoms of the illness under the same number of the needle insertions. Finally, one of the at least one set of the acupoints which has a largest weight in therapeutic effectiveness for the symptoms of the illness is output to the human machine interface 1 by the processing unit 3.

In the auxiliary medical system according to the invention, the storage unit 2 can further store the treatment result of the at least one symptom of the illness by each of the at least one set of the acupoints. The processing unit 3 can retrieve at least one set of the acupoints from the acupoint information which has a therapeutic effect for the at least one symptom of the illness and has the highest therapeutic effectiveness therefor. In this manner, the auxiliary medical system according to the invention is able to record the therapeutic effectiveness of each of the acupoints in the previous treatments. The recorded therapeutic effectiveness can be used for references in the future treatments.

In the auxiliary medical system according to the invention, the processing unit 3 can further mark the acupoints of the at least one set of the acupoints in the human body acupoint distribution image. The marked acupoints are displayed on the human machine interface 1. The approaches to mark the acupoints are of conventional knowledge and therefore are not elaborated for brevity.

Based on the above mechanism, during the use of the auxiliary acupuncture system according to an embodiment of the invention, the acupuncturist inquires the patient about the body condition and takes a pulse of the patient. According to the description made by the patient and the measured pulse, the acupuncturist determines and inputs at least one symptom of the illness to the human machine interface 1. Accordingly, the processing unit 3 retrieves at least one set of the acupoints from the acupoint information which has a therapeutic effect for the at least one symptom of the illness of the patient. The processing unit 3 also controls the human machine interface 1 to display the at least one set of the acupoints for the acupuncturist's selection. More specifically, the acupuncturist can input a medical requirement to the human machine interface 1 according to the requirement of the patient and/or the severities of different symptoms of the patient. For example, assume a patient has three symptoms “A,” “B” and “C,” the processing unit 3 can retrieve first, second and third sets of the acupoints which have a therapeutic effect for the symptoms “A,” “B” and “C.” In this regard, the first set of the acupoints is so determined that it must refer to one acupoint which has a therapeutic effect for all of the symptoms “A,” “B” and “C.” The second set of the acupoints is so determined that it must refer to two acupoints.

One of the two acupoints can be used for treatment for symptoms “A” and “B” with the effectiveness indices of 4 and 1, respectively. Another of the two acupoints can be used for treatment for symptoms “B” and “C” with the effectiveness indices of 3 and 2, respectively. The third set of the acupoints is so determined that it must refer to two acupoints. One of the two acupoints of the third set can be used for treatment for symptoms “A” and “B” with the effectiveness indices of 4 and 1, respectively. Another of the two acupoints of the third set can be used for treatment for symptoms “A,” “C” and “D” (symptom D is not on the patient) with the effectiveness indices of 2, 2 and 1, respectively.

According to the above, if the patient has a fear of acupuncture, the medical requirement may be the minimum number of the needle insertions, in order to reduce the pressure of the patient. In this regard, the processing unit 3 can retrieve one of the at least one set of the acupoints which meets the medical requirement of the minimum number of the needle insertions. Namely, the first set of the acupoints is retrieved and displayed on the human machine interface 1 for the acupuncturist's use.

Furthermore, assume symptom “A” of the patient has a higher degree of severity than the other symptoms. To deal with symptom “A” in a more efficient manner, the acupuncturist can retrieve one of the at least one set of the acupoints which has the highest therapeutic effectiveness for symptom “A.” In this regard, the processing unit 3 can calculate a total effectiveness index of each of the second and third sets of the acupoints in treatment for symptom “A.” After calculations, the total effectiveness indices of the second and third sets of the acupoints in treatment for symptom “A” are 4 and 6, respectively. Therefore, the processing unit 3 displays the third set of the acupoints on the human machine interface 1.

In another case, assume symptoms “A,” “B” and “C” have substantially the same degree of severity. To deal with all of the symptoms “A,” “B” and “C” in a more efficient manner, the acupuncturist can retrieve one of the at least one set of the acupoints which has the highest weight in therapeutic effectiveness for symptoms “A,” “B” and “C” under the same number of the needle insertions. In this regard, the processing unit 3 adds the effectiveness indices of those acupoints of the second set which have a therapeutic effect for symptoms “A,” “B” and “C” to yield a weight in therapeutic effectiveness, which is 10 in this example. Similarly, the processing unit 3 adds the effectiveness indices of those acupoints of the third set which have a therapeutic effect for symptoms “A,” “B” and “C” to yield a weight in therapeutic effectiveness, which is 9 in this example. As a result, the processing unit 3 displays the second set of the acupoints on the human machine interface 1.

In conclusion, the auxiliary medical system according to the invention is able to obtain at least one set of the acupoints, which has a therapeutic effect for at least one symptom of the illness, from the previously-established acupoint information. Each of the at least one set of the acupoints has a combination of the acupoints different from those of the other sets. Thus, the acupuncturist can select one of the at least one set of the acupoints to perform treatment for at least one symptom of the illness. Furthermore, the auxiliary medical system according to the invention permits the acupuncturist to perform different approaches of treatment on the patient through different combinations of the acupoints. As a result, low therapeutic effect resulting from the lack of the experience of the acupuncturist is avoided, thereby improving the medical quality.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. An auxiliary acupuncture system comprising:
a human machine interface configured to receive at least one symptom of an illness;
a storage unit configured to store acupoint information, wherein the acupoint information contains a name and a therapeutic function of each of a plurality of acupoints, and wherein the plurality of acupoints is divided into a plurality of sets of acupoints;
a processing unit coupled with the human machine interface and the storage unit, retrieving at least one of the plurality of sets of acupoints from the acupoint information which has a therapeutic effect for the at least one symptom of the illness, and displaying the at least one of the plurality of sets of acupoints on the human machine interface.

2. The auxiliary acupuncture system as claimed in claim 1, wherein the human machine interface further receives a medical requirement, and wherein the processing unit retrieves the at least one of the plurality of sets of acupoints which has the therapeutic effect for the at least one symptom of the illness and meets the medical requirement.

3. The auxiliary acupuncture system as claimed in claim 2, wherein the at least one symptom of the illness includes a plurality of symptoms of the illness, wherein the medical requirement indicates a minimum number of needle insertions, and wherein the processing unit selects and displays one of the at least one of the plurality of sets of acupoints which has a minimum number of acupoints on the human machine interface.

4. The auxiliary acupuncture system as claimed in claim 2, wherein the acupoint information contains an effectiveness index of each of the plurality of acupoints in treatment for each of the at least one symptom of the illness.

5. The auxiliary acupuncture system as claimed in claim 4, wherein the medical requirement indicates a highest therapeutic effectiveness for one of the at least one symptom of the illness under a same number of needle insertions, wherein each of the at least one of the plurality of sets of acupoints includes at least one of the plurality of acupoints which has the therapeutic effect for the symptom of the illness, wherein the effectiveness indices of the at least one of the plurality of acupoints are added to yield a sum for each of the at least one of the plurality of sets of acupoints, wherein the highest therapeutic effectiveness is the sum of one of the at least one of the plurality of sets of acupoints which is the largest, and wherein the one of the at least one of the plurality of sets of acupoints is output to the human machine interface.

6. The auxiliary acupuncture system as claimed in claim 4, wherein the at least one symptom of the illness includes a plurality of symptoms of the illness, wherein the medical requirement indicates a highest weight in therapeutic effectiveness for the plurality of symptoms of the illness under a same number of needle insertions, wherein each of the at least one of the plurality of sets of acupoints includes at least one of the plurality of acupoints, wherein the effectiveness indices of the at least one of the plurality of acupoints are added to yield a weight in therapeutic effectiveness for the plurality of symptoms of the illness for each of the at least one of the plurality of sets of acupoints, wherein the highest weight in therapeutic effectiveness is the weight in therapeutic effectiveness of one of the at least one of the plurality of sets of acupoints which is the highest, and wherein the one of the at least one of the plurality of sets of acupoints is output to the human machine interface.

7. The auxiliary acupuncture system as claimed in claim 1, wherein the storage unit further stores a human body acupoint distribution image, and wherein the processing unit marks the plurality of acupoints on the human body acupoint distribution image and displays the marked human body acupoint distribution image on the human machine interface.

8. The auxiliary acupuncture system as claimed in claim 2, wherein the storage unit further stores a human body acupoint distribution image, and wherein the processing unit marks the plurality of acupoints on the human body acupoint distribution image and displays the marked human body acupoint distribution image on the human machine interface.

9. The auxiliary acupuncture system as claimed in claim 3, wherein the storage unit further stores a human body acupoint distribution image, and wherein the processing unit marks the plurality of acupoints on the human body acupoint distribution image and displays the marked human body acupoint distribution image on the human machine interface.

10. The auxiliary acupuncture system as claimed in claim 4, wherein the storage unit further stores a human body acupoint distribution image, and wherein the processing unit marks the plurality of acupoints on the human body acupoint distribution image and displays the marked human body acupoint distribution image on the human machine interface.

11. The auxiliary acupuncture system as claimed in claim 5, wherein the storage unit further stores a human body acupoint distribution image, and wherein the processing unit marks the plurality of acupoints on the human body acupoint distribution image and displays the marked human body acupoint distribution image on the human machine interface.

12. The auxiliary acupuncture system as claimed in claim 6, wherein the storage unit further stores a human body acupoint distribution image, and wherein the processing unit marks the plurality of acupoints on the human body acupoint distribution image and displays the marked human body acupoint distribution image on the human machine interface.
